(19) United States
(12) Patent Application Publication
(10) Pub. No.: US 2024/0375964 A1
(43) Pub. Date: Nov. 14, 2024

(54) CONVERSION OF PHOTOSYNTHETICALLY DERIVED ORGANIC CARBON TO INORGANIC CARBON

(71) Applicant: EXXONMOBIL TECHNOLOGY AND ENGINEERING COMPANY, Annandale, NJ (US)

(72) Inventors: Giovanni PILLONI, Annandale, NJ (US); Leticia R. CARRILLO, ANNANDALE, NJ (US)

(21) Appl. No.: 18/657,451

(22) Filed: May 7, 2024

Related U.S. Application Data
(60) Provisional application No. 63/501,023, filed on May 9, 2023.

(51) Int. Cl. C01B 32/60; C12P 3/00

(52) U.S. Cl. CPC C01B 32/60; C12P 3/00

(57) ABSTRACT

Systems and methods are provided for integrated production of inorganic compounds from photosythetically-derived organic carbon. The photosynthetically-derived organic carbon can correspond to any convenient form of biomass and/or biologically formed products, such as amino acids. In some aspects, the biomass can include a combination of legumes and associated Rhizobia and/or ureolytic bacteria that can provide synergies for improved production of carbonates from the biomass

### CROSS REFERENCE TO RELATED APPLICATION

This application claims the benefit of U.S. Provisional Application No. 63/501,023, filed May 9, 2023, and titled “Conversion of Photosynthetically Derived Organic Carbon to Inorganic Carbon.”

### FIELD OF THE INVENTION

Systems and methods are provided for conversion of photosynthetically derived organic carbon compounds to inorganic carbon.

### BACKGROUND OF THE INVENTION

Photosynthesis is probably the most important biological processes on Earth, responsible for the conversion of solar energy into chemical energy, which in turns supports every form of life in the planet. Photosynthesis is performed by plants, algae, and certain bacteria. Additionally, the byproduct of photosynthesis, oxygen, is also essential for life on Earth.

Photosynthesis drives the conversion of roughly 20 gigatons per year of CO₂ into sequestered carbon, such as carbon in the form of biomass. This conversion is achieved even though about half of the CO₂ fixed by every plant is respired back by the plant itself, and despite the continuous biological degradation of plants' debris and roots' exudates by microorganisms.

For some of the carbon that is not respired back into the atmosphere, there is a mechanism known as “Microbial Precipitation of Calcium carbonate (MPC)”, a common phenomenon which converts CO₂ to more stable forms such as calcium carbonate found in marine waters, sediments, soil, and other environments. MPC can occur through two different mechanisms, either actively induced by bacteria or passively mediated by bacteria. The active mechanism is termed “Microbially Induced Calcium Carbonate (CaCO₃) Precipitation (MICP)” and involves microbial activities, such as urea hydrolysis, denitrification, sulfate reduction, and iron reduction. The passive mechanism occurs through the interaction of the organic matrix and calcium ions, without the necessity of direct biological activity but rather promoted by the presence of specific bacterial consortia. One of the advantages of MPC type processes is organic compounds formed via photosynthesis can be converted into inorganic compounds that are easier to use, sequester, or otherwise store in a form that does not readily return to the atmosphere.

It would be beneficial to have improved systems and/or methods for converting organic carbon compounds formed via photosynthesis into inorganic carbon compounds.

### SUMMARY

In an aspect, a method for forming metal carbonates from photosynthetically-derived carbon is provided. The method includes: growing a) at least one of legume biomass and cyanobacteria, and b) associated nodule-forming bacteria, ureolytic bacteria, or combination thereof, to generate organic carbon products comprising urea, ureides, or a combination thereof; converting at least a portion of the urea, ureides, or a combination thereof into conversion products comprising CO₂ and NH₃ at a pH of 7.5 to 10.0; and forming mineral carbonates from at least a portion of the CO₂.

### BRIEF DESCRIPTION OF THE DRAWINGS

FIG. 1 shows an integrated process flow for improving formation of carbonates from CO₂ and biomass.

### DETAILED DESCRIPTION

In various aspects, systems and methods are provided for integrated production of inorganic compounds (such as metal carbonates) from photosythetically-derived organic carbon. The photosynthetically-derived organic carbon can correspond to any convenient form of biomass and/or biologically formed products, such as amino acids. Preferably, the biomass can include a combination of legumes and associated Rhizobia and/or ureolytic bacteria that can provide synergies for improved production of carbonates from the biomass.

One of the difficulties with conventional methods for commercial and/or industrial conversion of carbon into inorganic forms (such as carbonates) is that all of the components of the conversion are brought together separately. In particular, the carbon for incorporation into mineral/inorganic form is typically from a first source, such as biomass that is brought in and/or CO₂ that is sequestered specifically for the incorporation. The bacteria/microbes needed for conversion of the carbon are then separately added to the environment, along with any other needed reagents. The ratios of added materials are then adjusted to achieve desired reaction conditions.

In various aspects, systems and methods are provided for integration of production of organic carbon for sequestration with conversion of the organic carbon to inorganic/mineral form. This is achieved by using legumes and/or cyanobacteria as the photosynthetic source, in combination with Rhizobia bacteria, or another type of bacteria with similar functionality. Rhizobia bacteria can form nodules in combination with the root system of legumes, so that the Rhizobia can interact symbiotically with the legume biomass. The Rhizobia can fix nitrogen to form ammonia, which is provided to the legume. The legume returns nutrients, including ureides and amino acids, to the nodule.

In various aspects, the nodules associated with the legume root system can be engineered to provide urease and/or an increased amount of urease. The urease can be used to break down urea and/or other ureides to form ammonia and carbamic acid, which can then decompose to form additional ammonia and CO₂. Additionally or alternately, ureolytic bacteria can be added to the soil environment to aid in the process of breaking down urea and/or ureides. The ammonia formed during the reactions provides a basic environment that converts CO₂ into bicarbonate or carbonate that can then be combined with a counterion (or counterions) to form a mineral carbonate. The conversion to mineral carbonate is facilitated by bacteria in the soil environment. Optionally, the Rhizobia bacteria and/or the ureolytic bacteria can facilitate this conversion.

The mutualistic symbiotic relationship between the plant family Leguminosae (Fabaceae) and a group of soil bacteria called Rhizobia have evolved to benefit each other by exchange of ammonium for reduced carbon compounds and other nutrients. Rhizobia infect the roots of the legumes creating nodules before differentiating into bacteroids (the symbiotic form of rhizobia). The symbiotic relationship is rather complex and involves the supply of C4-dicarboxylate and phosphate from the host plant to the bacterioids, that utilize them for the energy-intensive process of fixing nitrogen into ammonium, which is a source of macronutrient for the plant. For the passive pathways optimization of ureolytic bacteria, such as those containing the enzyme microbial urease, or a consortium of these types of microorganisms, can catalyze the hydrolysis of urea into ammonia and carbamic acid. Through a series of spontaneous chemical reactions and an increased pH within the microenvironment allow for optimal basis for CaCO₃ precipitation. Furthermore, the presence of functional groups such as carboxylic acids (R-COOH), hydroxyl groups (R—OH), amino groups (R—NH2), sulfate-(R—O—SO3H), and sulfhydryl groups (—SH), deprotonate due to an increase in pH, which results in an overall negative charge of extrapolysaccharides produced by the cell, which facilitates its binding to metal ions.

By modifying Rhizobia nodules to provide increased amounts of urease, the organic carbon for sequester in mineral form can be provided in situ for the bacteria that performs the mineralization. This corresponds to a first pathway for converting organic carbon formed by photosynthesis into inorganic mineral carbon.

In addition to providing urease, the nodules also provide a pathway for introducing amino acids into the soil environment. These amino acids can be decarboxylated to form CO₂ and other products, such as ammonia and water. This provides a second source of CO₂ for conversion into mineral carbonates, along with additional ammonia to contribute to the basic soil environment.

The formation of mineral carbonates can be further enhanced by addition of carbonic anhydrase to the soil environment. Carbonic anhydrase provides an alternative route for formation of mineral carbonates from CO₂, separate from bacterial conversion. Carbonic anhydrase can be provided to the soil environment by various types of bacteria and/or microbes. In some aspects, the Rhizobia and/or ureolytic bacteria can provide carbonic anhydrase for the soil environment. In some aspects, microbes for providing carbonic anhydrase can be part of the nodules associated with legume and/or cyanobacteria used for forming the photosynthetically-derived organic compounds.

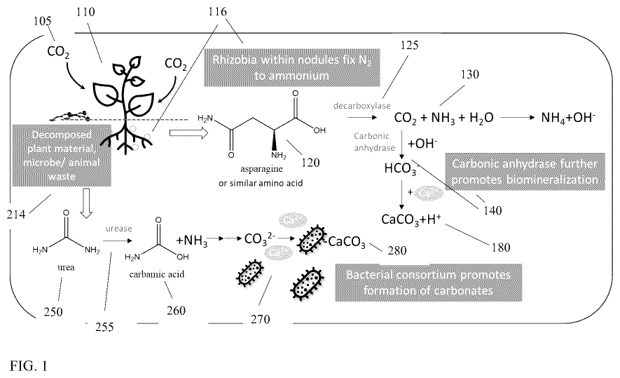

FIG. 1 shows an example of the integration of multiple pathways for mineralization of carbon. In FIG. 1, CO₂ 105 is consumed, in conjunction with light (not shown) to form additional biomass 110. CO₂ 105 can be provided from any convenient source. In some aspects, CO₂ 105 can correspond to CO₂ from a low concentration source, such as the roughly 400 ppm CO₂ that is typically present in air. In some aspects, such as aspects where a greenhouse or other controlled environment is used for growing biomass 110, the CO₂ 105 can correspond to CO₂ captured from a CO₂ source, such as CO₂ captured from an industrial flue gas and/or a combustion flue gas. In such aspects, higher CO₂ concentrations can be available. Biomass 110 can correspond to any convenient type of biomass, such as plants, bacteria, or other micro-organisms. In the scheme shown in FIG. 1, biomass can be mineralized by multiple complementary pathways in order to increase the amount of mineralization relative to the amount of biomass 110.

One pathway corresponds to conversion of urea into mineral carbonates in the presence of one or more types of bacteria that can provide microbial urease. Urea 250 can be formed 214 either as a waste product from living biomass or as a decomposition product as biomass decomposes. It is noted that urea from animal waste could also be provided to the environment. A variety of bacteria can produce urease 255 that can convert urea 250 into carbamic acid plus ammonia. Further bacterial processes 270 can then be used to convert the carbamic acid into mineral carbonates 280. The further bacterial processes 270 can take place in the presence of suitable pH values and a suitable concentration of metals/metal ions for forming the mineral carbonates. In some aspects, the Rhizobia and/or ureolytic bacteria can correspond to the bacteria that convert the carbamic acid into mineral carbonates 280.

It is noted that although urea is shown as the compound converted to form mineral carbonates 280, CO₂ from any source can be converted. For example, CO₂ formed from decarboxylation of amino acids can also be converted to mineral carbonates.

In addition to having an additional source of carbon, FIG. 1 also shows an alternative pathway for conversion of CO₂ to mineral carbonates. As shown in FIG. 1, decarboxylation 125 of an organic substrate such as an amino acid, can be performed to form reaction products 130, including CO₂ and ammonia (and optionally water). Carbonic anhydrase 140 can then be used to convert CO₂ into bicarbonate or carbonate. The bicarbonate and/or carbonate can then combine with metals such as sodium, calcium, magnesium, and/or potassium to form metal carbonates. It is noted that CO₂ formed via the urease pathway can also be converted to mineral carbonates using carbonic anhydrase.

In addition to increasing the amount of organic carbon that can be converted to carbonates, the addition of legume-based biomass and Rhizobia to the mineralization environment provides opportunities for synergies between the different mineralization pathways. For example, the conversion of CO₂ to carbonate or bicarbonate using carbonic anhydrase benefits from having an excess of hydroxyl ions present. The pathway for decarboxylation of amino acids provides some ammonia, but additional ammonia is generated from the breakdown of urea using urease. The additional ammonia can further increase the pH in the reaction environment for the carbonic anhydrase, thus facilitating the formation of bicarbonate for combination with metals.

While FIG. 1 shows a general overview of the integrated process, a number of additional improvements are also contemplated.

### Improvements for Root Nodules

A first class of improvements to the general systems and/or methods can be to engineer Rhizobia (and/or other bacteria) to provide increased production of ureides. By increasing the availability of urea and/or ureides, the amount of mineralized carbonates can be increased.

As second type of improvement can be engineering of an improved consortia of bacteria for conversion of urea and/or ureides to mineral carbonates.

A third type of improvement can be engineering of bacteria that serve as the nucleation sites for formation of mineral carbonates.

### Improvements for Soil Environment

The conditions for formation of mineral carbonates can also be improved.

Examples of the conditions that can be modified include, but are not limited to, pH; dissolved inorganic carbon (DIC) concentrations; calcium concentrations; urea and/or ureide concentration; carbonic anhydrase concentration; and availability of nucleation sites.

In various aspects, the pH of the soil environment during formation of mineral carbonates can be maintained at a pH between 7.5 to 10.0, or 7.5 to 9.5, or 7.5 to 9.0, or 8.0 to 10.0, or 8.0 to 9.5, or 8.0 to 9.0, or 8.5 to 10.0, or 8.5 to 9.5. The production of ammonia during conversion of photosynthetically-produced carbon compounds can tend to increase the pH of the soil. Thus, in some aspects, addition of acid to the soil environment can be used to control the pH to a desired level.

In various aspects, the dissolved inorganic carbon concentration in the soil can be between 10 mM to 50 mM, or 10 mM to 35 mM, or 10 mM to 25 mM, or 20 mM to 50 mM.

In various aspects, the concentration of alkali metals, alkaline earth metals, and/or iron in the soil can be between 10 mM to 100 mM, or 10 mM to 50 mM, or 10 mM to 35 mM, or 20 mM to 100 mM, or 20 mM to 50 mM, or 35 mM to 100 mM. In some aspects, the metal can be calcium. In some aspects, the metal can be one or more of sodium, potassium, calcium, iron, and magnesium.

In various aspects, the concentration of urea and/or ureides in the soil can be between 10 mM to 50 mM, or 10 mM to 35 mM, or 10 mM to 25 mM, or 20 mM to 50 mM.

In various aspects, the carbonic anhydrase concentration in the soil can be between 10 mM to 50 mM, or 10 mM to 35 mM, or 10 mM to 25 mM, or 20 mM to 50 mM. Carbonic anhydrase is a zinc containing metallo-enzyme that catalyzes the reverse hydration of CO₂ to bicarbonate and is ubiquitous in prokaryotes and eukaryotes.

In various aspects, the quantity of nucleation sites in the soil can be 10 mM to 50 mM, or 10 mM to 35 mM, or 10 mM to 25 mM, or 20 mM to 50 mM. The quantity of nucleation sites can be correlated with the quantity of bacteria in the soil.

### Additional Embodiments

Embodiment 1. A method for forming metal carbonates from photosynthetically-derived carbon, comprising: growing a) at least one of legume biomass and cyanobacteria, and b) associated nodule-forming bacteria, ureolytic bacteria, or combination thereof, to generate organic carbon products comprising urea, ureides, or a combination thereof; converting at least a portion of the urea, ureides, or a combination thereof into conversion products comprising CO₂ and NH₃ at a pH of 7.5 to 10.0; and forming mineral carbonates from at least a portion of the CO₂.

Embodiment 2. The method of Embodiment 1, wherein forming mineral carbonates comprises reacting the at least a portion of the CO₂ in the presence of at least one mineral-forming bacteria and one or more of Na, K, Ca, Mg, and Fe, the mineral-forming bacteria optionally comprising the associated nodule-forming bacteria, ureolytic bacteria, or combination thereof.

Embodiment 3. The method of any of the above embodiments, wherein the concentration of urea, ureides, or a combination thereof is 10 mM to 50 mM.

Embodiment 4. The method of any of the above embodiments, wherein the concentration of the one or more of Na, K, Ca, Mg, and Fe is 10 mM to 100 mM.

Embodiment 5. The method of any of the above embodiments, wherein forming mineral carbonates comprises reacting the at least a portion of the CO₂ in the presence of carbonic anhydrase, the concentration of carbonic anhydrase optionally being 10 mM to 50 mM.

Embodiment 6. The method of any of the above embodiments, wherein the nodule-forming bacteria comprise Rhizobia.

Embodiment 7. The method of any of the above embodiments, wherein growing the at least one of legume biomass and cyanobacteria further comprises forming one or more amino acids, and wherein the converting further comprises converting at least a portion of the one or more amino acids.

Embodiment 8. The method of any of the above embodiments, wherein the associated nodule-forming bacteria, ureolytic bacteria, or combination thereof provides urease for the converting.

While the present invention has been described and illustrated by reference to particular embodiments, those of ordinary skill in the art will appreciate that the invention lends itself to variations not necessarily illustrated herein. It will also be apparent to those skilled in the art that when numerical lower limits and numerical upper limits are listed herein, ranges from any lower limit to any upper limit are contemplated. Also, all numerical values within the detailed description herein are modified by “about” the indicated value, and take into account experimental error and variations that would be expected by a person having ordinary skill in the art. Where in the foregoing description, integers or elements are mentioned which have known, obvious or foreseeable equivalents, then such equivalents are herein incorporated as if individually set forth. For these reasons, then, reference should be made solely to the appended claims for purposes of determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

What is claimed is:

1. A method for forming metal carbonates from photosynthetically-derived carbon, comprising:
growing a) at least one of legume biomass and cyanobacteria, and b) associated nodule-forming bacteria, ureolytic bacteria, or combination thereof, to generate organic carbon products comprising urea, ureides, or a combination thereof;
converting at least a portion of the urea, ureides, or a combination thereof into conversion products comprising CO₂ and NH₃ at a pH of 7.5 to 10.0; and
forming mineral carbonates from at least a portion of the CO₂.

2. The method of claim 1, wherein forming mineral carbonates comprises reacting the at least a portion of the CO₂ in the presence of at least one mineral-forming bacteria and one or more of Na, K, Ca, Mg, and Fe.

3. The method of claim 2, wherein the mineral-forming bacteria comprises the associated nodule-forming bacteria, ureolytic bacteria, or combination thereof.

4. The method of claim 1, wherein the concentration of urea, ureides, or a combination thereof is 10 mM to 50 mM.

5. The method of claim 1, wherein the concentration of the one or more of Na, K, Ca, Mg, and Fe is 10 mM to 100 mM.

6. The method of claim 1, wherein forming mineral carbonates comprises reacting the at least a portion of the CO₂ in the presence of carbonic anhydrase.

7. The method of claim 6, wherein the concentration of carbonic anhydrase is 10 mM to 50 mM.

8. The method of claim 1, wherein the nodule-forming bacteria comprise Rhizobia.

9. The method of claim 1, wherein growing the at least one of legume biomass and cyanobacteria further comprises forming one or more amino acids, and wherein the converting further comprises converting at least a portion of the one or more amino acids.

10. The method of claim 1, wherein the associated nodule-forming bacteria, ureolytic bacteria, or combination thereof provides urease for the converting.
